(19) United States
(12) Patent Application Publication
(10) Pub. No.: US 2019/0110997 A1
(43) Pub. Date: Apr. 18, 2019

(54) ORAL ADMINISTRATION FORM COMPRISING PROBIOTIC BACTERIA

(71) Applicant: Merck Patent GmbH, Darmstadt (DE)

(72) Inventors: Markus RUDOLPH, Neu-Isenburg (DE); Stefan HENKE, Kirchen (DE); Iris MANNECK, Darmstadt (DE)

(73) Assignee: Merck Patent GmbH, Darmstadt (DE)

(21) Appl. No.: 16/193,470

(22) Filed: Nov. 16, 2018

Related U.S. Application Data
(63) Continuation of application No. 11/597,514, filed on Nov. 27, 2006, now abandoned.

(30) Foreign Application Priority Data
May 28, 2004 (DE) 10 2004 026 706.5

(51) Int. Cl. A61K 9/20

(52) U.S. Cl. CPC A61K 9/2054; A61K 9/2013; A61K 9/2009

(57) ABSTRACT

The invention relates to an oral administration form which comprises at least one species of probiotic microorganisms, where it itself and/or the probiotic bacteria is/are provided with a coating which comprises at least two cellulose ethers.

The invention relates to an oral administration form which comprises at least one species of probiotic microorganisms, where it itself and/or the probiotic bacteria is/are provided with a coating which comprises at least two cellulose ethers.

Probiotic microorganisms are already employed diversely today in the form of selected foods, food supplement preparations or medicaments in order to ameliorate or eliminate symptoms which causes disturbed or damaged intestinal flora. One problem is the high loss of activity of the probiotic microorganisms of about 97% of the initial value at the end of the small intestine on oral administration. It is therefore necessary to provide a significantly larger amount of the probiotic microorganisms in order to achieve adequately high activity.

DE 1937361 A1 describes an oral administration form comprising probiotic microorganisms in which the loss of activity of the probiotic microorganisms which is associated with the stomach-intestine passage is claimed to be prevented by the use of a gastric juice-resistant coating consisting of shellac. It is disadvantageous in this administration form that the dissolution of the coating material dependent on a plurality of physiological conditions, such as the intraluminal pH in the gastrointestinal tract, the administration conditions (pre-, postprandial or with a meal), the composition of the meal, the age of the user, diseases, amount of liquid and the simultaneous administration of medicaments, such as, for example, antacids. Furthermore, the use and processing of shellac is not unproblematical since shellac is a natural product an, as a consequence of the natural variations in its composition associated therewith, is not always available in the constant quality necessary for reproducible dissolution behaviour. In the case of increased moisture levels, sticking together can occur in the case of shellac film tablets, meaning that the integrity of the coating may be impaired. Compliance of the user and the efficacy of the probiotic microorganisms are thus possibly not fully guaranteed. Furthermore, shellac can only be processed with organic solvents, which results in increased costs compared with processing of aqueous solutions and may disadvantageously result in residues of organic solvents, which are undesired from a toxicological point of view, remaining in the administration form. In addition, the use of shellac as coating material requires larger amounts of a softening additive since shellac is unsuitable as coating material without the addition of softeners as a consequence of its very high brittleness and fragility. However, the addition of softeners is problematical since they may escape into the environment from the shellac film during storage of the finished administration form, impairing the properties of the coating and shortening the shelf life of the administration form. In addition, shellac “ages” during storage, i.e. the functional groups present in the shellac may react with one another and thus crosslink, which results in a slowing of the dissolution time of the shellac coating.

The object of the present invention was to provide an oral administration form which liberates the probiotic microorganisms reproducibly in the human and/or animal intestine in order to ensure the activity of the probiotic microorganisms and thus health-promoting action thereof in the intestine. The oral administration form should furthermore have a good shelf life and be simple and inexpensive to produce.

Surprisingly, the object has been achieved by the provision of an oral administration form which comprises at least one species of probiotic microorganisms and which itself and/or in which the probiotic microorganisms is/are provided with a coating which comprises at least two cellulose ethers. The invention therefore relates to an oral administration form comprising at least one species of probiotic microorganisms which is characterised in that it itself and/or in which the probiotic microorganisms is/are provided with a coating which comprises at least two cellulose ethers.

The coating comprising at least two cellulose ethers can be applied from aqueous solutions, meaning that residues of organic solvents can basically be avoided. The addition of softeners in the coating can advantageously be avoided, meaning that the storage stability is not impaired thereby.

The oral administration form is preferably a tablet, a dragee, a capsule, a granular material, a pellet preparation or a powder, particularly preferably a tablet and very particularly preferably a multilayered tablet.

Suitable probiotic microorganisms are all microorganisms which either themselves usually occur in the healthy human or animal body and have a health-promoting action on the healthy, unhealthy or diseased human or animal body.

The probiotic microorganisms employed are preferably living Lactobacilli, Bifidobacteria and/or Streptococci. Particular preference is given to the species Lactobacillus casei, Lactobacillus acidophilus, Lactobacillus reuteri, Lactobacillus bifidum, Lactobacillus gasseri, Lactobacillus plantarum, Lactobacillus johnsonii, Lactobacillus rhamnosus, Lactobacillus fermentum, Lactobacillus paracasei, Lactobacillus crispatus, Bifidobacterium longum, Bifidobacterium bifidum, Bifidobacterium longum, Bifidobacterium lactis, Bifidobacterium brevis, Bifidobacterium animalis, Bifidobacterium adolescentis, Bifidobacterium infantis, Streptococcus thermophilus and/or Lactococcus lactis.

The amount of the probiotic microorganisms in the oral administration form according to the invention should be selected in such a way that the health-promoting action aimed at is ensured. The oral administration form according to the invention preferably comprises 10³ to 10¹², particularly preferably 10⁵ to 10¹¹ and very particularly preferably 10⁷ to 10¹⁰ probiotic microorganisms. It is advantageous for the stability with respect to the number and activity of living microorganisms if the materials used, in particular the support material in which the probiotic microorganisms are embedded, have the lowest possible water content. The water content is preferably ≤3.0% by weight, particularly preferably ≤0.1% by weight, based on the weight of the support material.

The cellulose ethers present in the coating of the oral administration form according to the invention are substances which are swellable or form a gel in aqueous media, where the swelling or gel formation takes place to different extents and at different rates depending on the ether substituents present in the respective cellulose ether. The coating of the oral administration form according to the invention comprises at least two cellulose ethers, each having different swelling or gel-formation behaviour, which are matched to one another in such a way that the probiotic cultures present in the oral administration form according to the invention are released in a delayed manner in the intestine after the oral administration form has been taken by the user as a consequence of swelling and ultimately dissolution and/or structural detachment of the coating. The delay in the release is substantially independent of the respective pH conditions and is such that it corresponds to the time that the oral administration form requires in order to pass through the stomach in substantially unchanged form and to reach the intestine after being taken by the user. Suitable delay times for the oral administration form according to the invention are those which increase the survival rate of the probiotic microorganisms in the small intestine as far as the terminal ileum by at least 5-fold compared with the uncoated administration form. The duration of the delay is dependent on the type of cellulose ethers present in the coating and can be adjusted to the desired value by variation of the mixing ratios in which they are present with respect to one another and by variation of the layer thickness of the coating. The mixing ratios of the cellulose ethers to one another which are necessary in each case for the desired release profile and the layer thickness which is necessary in each case can be determined and optimised here with reference to experiments in in-vitro models, for example the so-called TNO model (dynamic gastrointestinal model, as described in Marteau, P et al. (1997) Survival of Lactic Acid Bacteria in a Dynamic Model of the Stomach and Small Intestine: Validation and the Effects of Bile, J Dairy Sci 80:1031-1037).

In general, a coating comprising two different cellulose ethers comprises the latter in a weight ratio of 0.1:99.9 to 99.9:0.1. The layer thickness of the coating is generally 0.5 to 20 mg per cm², preferably 5 to 15 mg per cm².

According to an embodiment of the invention, the oral administration form comprises in the coating cellulose ethers which contain, as ether substituents, hydroxyalkyl groups, preferably hydroxyethyl, hydroxypropyl and/or dihydroxypropyl groups, particularly preferably hydroxypropyl groups. Cellulose ethers containing hydroxyalkyl groups as ether substituents which can be employed for the invention are accordingly, for example, hydroxyethylcellulose, hydroxypropylcellulose and di-hydroxypropylcellulose.

According to a preferred embodiment of the invention, at least one of the cellulose ethers present in the coating of the oral administration form also contains alkyl groups, preferably methyl and/or ethyl groups, particularly preferably methyl groups, as ether substituents besides hydroxyalkyl groups. Suitable cellulose ethers for the invention which also contain alkyl groups as ether substituents besides hydroxyalkyl groups are, for example, ethylhydroxyethylcellulose, hydroxypropylmethylcellulose, hydroxypropylethylcellulose and hydroxyethylmethylcellulose.

According to a particularly preferred embodiment of the invention, the coating of the oral administration form according to the invention comprises one cellulose ether which contains exclusively hydroxyalkyl groups as ether substituents together with one cellulose ether which also contains alkyl groups as ether substituents besides hydroxyalkyl groups.

The oral administration form according to the invention very particularly preferably comprises hydroxypropylmethylcellulose and hydroxypropylcellulose as cellulose ethers in the coating. Hydroxypropylmethylcellulose and hydroxypropylcellulose may be present here in a weight ratio to one another of 90:10 to 10:90, preferably in a weight ratio to one another of 30:70 to 70:30, particularly preferably in a weight ratio of about 35:65. Hydroxypropylmethylcellulose and hydroxypropylcellulose are preferably employed as a binary mixture.

The proportion by weight of the cellulose ethers present in the coating, based on the total weight of the oral administration form, is preferably 1 to 20% by weight, particularly preferably 1.5 to 10% by weight and very particularly preferably 3 to 5% by weight.

Besides the cellulose ethers mentioned, the coating may also, for example in order to increase its physical stability, comprise alkylcellulose ethers, such as, for example, methylcellulose or ethylcellulose. If alkylcellulose ethers are present, they are preferably present in an amount of 0.5 to 10% by weight, based on the dry weight of the coating.

It is essential for the oral administration form according to the invention that it is completely surrounded by the coating.

A further preferred embodiment of the oral administration form comprises probiotic microorganisms which are themselves provided with a gastric juice-resistant coating. To this end, the probiotic microorganisms are dried by various methods known to the person skilled in the art and subsequently provided with the coating.

The coating preferably does not comprise any further adjuvants. On a production scale, it may be helpful to employ a release agent. Preference is then given to the use of stearates, for example magnesium stearate, glycerol monostearate, glyceryl dipalmitostearate or talc. Stearates may be present in a proportion by weight of 1 to 10% by weight, based on the dry weight of the coating, preference is given to about 5% by weight of talc, based on the dry weight of the coating, in a proportion by weight of up to 100% by weight, preferably a proportion by weight of 30 to 50% by weight.

The coating can be applied from aqueous, organic or hydroalcoholic solution. The coating is preferably applied from aqueous solution. The invention therefore also relates to a process for the production of the oral administration form according to the invention which is characterised in that the coating is applied from aqueous solution and/or from organic solution, preferably from aqueous solution. In the case of application of the coating by means of an organic solution, this is preferably carried out from alcoholic solution, particularly preferably from hydroalcoholic solution, i.e. from a mixture of water and alcohol. The alcohol employed is preferably ethanol.

The coating can be applied by conventional methods known to the person skilled in the art, such as, for example, tablet coating, spraying of solutions, dispersions or suspensions, by melt methods or by powder application methods. The coating is preferably applied by means of a drum coater or by the fluidised-bed method, for example by the Wurster method.

The coatings appear clear to opaque. For colouring, coloured pigments, lakes or dyes can be added.

According to a preferred embodiment, the oral administration form according to the invention comprises further nutrition-relevant additives in addition to the probiotic microorganisms. It preferably comprises vitamins, mineral substances, trace elements, roughage, enzymes, plant extracts, proteins, carbohydrates and/or fats. If the oral administration form comprises nutrition-relevant additives whose digestion already begins in the stomach, such as, for example, proteins, it is important that these nutrition-relevant additives are at least incompletely surrounded by the coating.

Depending on the nutrition-relevant additives employed here, it may be necessary to incorporate them into the oral administration form according to the invention in such a way that they do not come into contact with one another and/or with the probiotic microorganisms. This is preferably achieved by incorporation of the nutrition-relevant additives and/or microorganisms into different layers of a multilayered tablet.

Preferred vitamins are vitamin A (β-carotene), vitamin C, vitamin E, vitamins of the B complex and/or vitamin K. Particularly preferred vitamins are vitamin A, vitamin C and/or vitamin E. The amount of vitamins generally depends on the recommended minimum required dose for the respective vitamin, but this may also be exceeded by on average 50 to 300%. Preferred ranges are between 50 and 300 mg for vitamin C, 10 to 50 mg for vitamin E, ≤1.5 mg for vitamin A and 10 μg to 20 mg for the vitamins of the B complex.

Preferred mineral substances are inorganic or organic sodium, potassium, calcium, magnesium, zinc and/or iron salts which are suitable for consumption, preferably in the form of carbonates, bicarbonates, phosphates, biphosphates, sulfates, bisulfates, chlorides, fluorides, citrates and/or lactates. The proportion of mineral substances, based on the total weight of the oral administration form, is preferably from 20 to 40% by weight. The oral administration form according to the invention preferably comprises silicon, chromium, manganese, iodine, molybdenum and/or selenium as trace elements.

As roughage, the oral administration form according to the invention preferably comprises soya bran, maize bran, wheat bran and/or cereal whole grain, particularly preferably soya bran. The proportion of roughage, based on the total weight of the oral administration form, is preferably 2 to 50% by weight.

Preferred enzymes or coenzymes are lipases and/or proteases or CoEnzym Q, superoxide dismutase and/or gluthathione peroxidase, which promote stomach and/or intestinal function and/or metabolism. These can be introduced in an amount known per se and in a form known per se.

The oral administration form may additionally comprise further probiotic substances, preferably oligofructose and/or other oligo sugars.

Preferred plant extracts are dry extracts and here in particular those which comprise bioflavonoids, polyphenols, phytooestrogens and/or saponins, such as, for example, from Echinaceae.

The oral administration form according to the invention preferably comprises, as proteins, soya protein and/or milk protein and/or, as fats, fats which contain polyunsaturated fatty acids.

The oral administration form according to the invention may additionally comprise conventional adjuvants and additives, depending on the embodiment. The choice of adjuvants and/or additives also depends on the food regulations of the country in which the oral administration form according to the invention is to be used. The adjuvants and/or additives used, for example for the tablets, multilayered tablets and/or dragees according to the invention, are starch (for example corn starch), talc, microcrystalline cellulose, lactose, highly disperse silicon dioxide, polyvinylpyrrolidone and/or cellulose powder. Further constituents which can be employed as binders and/or release agents are carbohydrates, such as, for example, mannitol, sorbitol, xylitol, glucose, sucrose, fructose, maltose, dextrose, maltodextrin and/or kaolin, and/or cellulose derivatives, such as, for example, methylcellulose, hydroxypropylcellulose and/or hydroxypropylmethylcellulose, and/or calcium carbonate, calcium stearate, magnesium stearate and/or glycerol stearate. The oral administration form according to the invention may furthermore also comprise dyes, flavours and/or aromas, as well as lubricants, antioxidants and/or stabilisers. The content of these basic substances depends on the one hand on the target content of probiotic microorganisms, vitamins, enzymes, roughage, etc. and on the other hand on criteria which determine the mechanical-physical properties of the oral administration form, such as, for example, hardness, compressibility, size, colour and/or shape.

The oral administration form according to the invention can be produced by various methods known to the person skilled in the art. These methods are disclosed, for example, in H. Sucker, P. Fuchs, P. Speiser, “Pharmazeutische Technologie” [Pharmaceutical Technology], Stuttgart 1978 or K. H. Bauer, K. H. Frömming, C. Führer, “Pharmazeutische Technologie” [Pharmaceutical Technology], Stuttgart 1986. They are hereby incorporated by way of reference and are thus part of the disclosure.

The examples explain the invention without being restricted thereto.

### EXAMPLE 1

A mixture of 45% of bacteria preparation, 28.7% of tricalcium phosphate, 19% of microcrystalline cellulose, 2% of glyceryl palmitostearate, 0.6% of magnesium stearate and 4.7% of disintegrant was compressed together with a vitamin and mineral substance mixture in an E. Hata rotary tablet press to give a bean-shaped tablet having a core weight of 1.0 g and the dimensions 18.0 mm×8.0 mm×7.2 mm. A mixture of 35 parts of hydroxypropylmethylcellulose and 65 parts of hydroxypropylcellulose was subsequently sprayed on from aqueous solution in an O'Hara drum coater with a batch size of 15 kg. The amount of sprayed-on hydroxypropylmethylcellulose/hydroxypropylcellulose mixture was 5% by weight, based on the weight of the core, corresponding to 11.74 mg per cm² of tablet surface.

In an in-vitro experiment in the TNO model, the uncoated tablet cores are compared with the coated tablets from Example 1. The recovery from the initial value of 100% represents the survival rate of the probiotic microorganisms after passing through the stomach and small intestine model.

Survival rate of
Bifidobacteria

| Passage time (min) | Tablet core | Coated tablet |
|---|---|---|
| 360 | 2.3% | 21.2% |

### EXAMPLE 2

A mixture of 65% of bacteria preparation, 20% of tricalcium phosphate, 6% of microcrystalline cellulose, 2% of glyceryl palmitostearate, 0.6% of magnesium stearate and 6.4% of disintegrant was compressed together with a vitamin and mineral substance mixture in a Fette rotary tablet press to give a bean-shaped tablet having a core weight of 1.35 g and the dimensions 21.0 mm×10.0 mm×8 mm. A mixture of 50 parts of hydroxypropylmethylcellulose and 50 parts of hydroxypropylcellulose was subsequently sprayed on from aqueous solution in an O'Hara drum coater with a batch size of 15 kg. The amount of sprayed-on hydroxypropylmethylcellulose/hydroxypropylcellulose mixture was 7% by weight, based on the weight of the core, corresponding to 17.42 mg per cm² of tablet surface.

### EXAMPLE 3

A mixture of 45% of bacteria preparation, 28.7% of tricalcium phosphate, 19% of microcrystalline cellulose, 2% of glyceryl palmitostearate, 0.6% of magnesium stearate and 4.7% of disintegrant was compressed together with a vitamin and mineral substance mixture in an E. Hata rotary tablet press to give a bean-shaped tablet having a core weight of 1.0 g and the dimensions 18.0 mm×8.0 mm×7.2 mm. A mixture of 35 parts of hydroxypropylmethylcellulose and 65 parts of hydroxypropylcellulose was subsequently sprayed on from aqueous solution in a Pellegrini drum coater with a stomach size of 250 kg. The amount of sprayed-on hydroxypropylmethylcellulose/hydroxypropylcellulose mixture was 5% by weight, based on the weight of the core, corresponding to 11.74 mg per cm² of tablet surface.

In an in-vitro experiment in the TNO model, the uncoated tablet cores are compared with the coated tablets from Example 3. The recovery from the initial value of 100% represents the survival rate of the probiotic microorganisms after passing through the stomach and small intestine model.

Survival rate of
Lactobacillus

| Passage time (min) | Tablet core | Coated tablet |
|---|---|---|
| 360 | 1.6% | 9.7% |

### EXAMPLE 4

A mixture of 65% of bacteria preparation, 20% of tricalcium phosphate, 6% of microcrystalline cellulose, 2% of glyceryl palmitostearate, 0.6% of magnesium stearate and 6.4% of disintegrant was compressed together with a vitamin and mineral substance mixture in a Fette rotary tablet press to give a bean-shaped tablet having a core weight of 1.35 g and the dimensions 21.0 mm×10.0 mm×8 mm. A mixture of 35 parts of hydroxypropylmethylcellulose and 65 parts of hydroxypropylcellulose was subsequently sprayed on from aqueous solution in an O'Hara drum coater with a stomach size of 15 kg. The amount of sprayed-on hydroxypropylmethylcellulose/hydroxypropylcellulose mixture was 7% by weight, based on the weight of the core, corresponding to 17.42 mg per cm² of tablet surface.

### EXAMPLE 5

A mixture of 45% of bacteria preparation, 28.7% of tricalcium phosphate, 19% of microcrystalline cellulose, 2% of glyceryl palmitostearate, 0.6% of magnesium stearate and 4.7% of disintegrant was compressed together with a vitamin and mineral substance mixture in an E. Hata rotary tablet press to give a bean-shaped tablet having a core weight of 1.0 g and the dimensions 18.0 mm×8.0 mm×7.2 mm. A mixture of 35 parts of hydroxypropylmethylcellulose and 65 parts of hydroxypropylcellulose was subsequently dissolved in water, and 5% by weight of magnesium stearate, based on the polymer dry substance, were subsequently incorporated, and the mixture was sprayed on in a Pellegrini drum coater with a stomach size of 250 kg. The amount of sprayed-on hydroxypropylmethylcellulose/hydroxypropylcellulose mixture was 5% by weight, based on the weight of the core, corresponding to 11.74 mg per cm² of tablet surface.

### EXAMPLE 6

A mixture of 45% of bacteria preparation, 28.7% of tricalcium phosphate, 19% of microcrystalline cellulose, 2% of glyceryl palmitostearate, 0.6% of magnesium stearate and 4.7% of disintegrant was compressed together with a vitamin and mineral substance mixture in an E. Hata rotary tablet press to give a bean-shaped tablet having a core weight of 1.0 g and the dimensions 18.0 mm×8.0 mm×7.2 mm. A mixture of 35 parts of hydroxypropylmethylcellulose and 65 parts of hydroxypropylcellulose was subsequently dissolved in an ethanol/water mixture (70 parts:30 parts), and 5% by weight of magnesium stearate, based on the polymer dry substance, were subsequently incorporated, and the mixture was sprayed on in a Pellegrini drum coater with a stomach size of 333 kg. The amount of sprayed-on hydroxypropylmethylcellulose/hydroxypropylcellulose mixture was 7% by weight, based on the weight of the core, corresponding to 16.43 mg per cm² of tablet surface.

### EXAMPLE 7

A mixture of 45% of bacteria preparation, 28.7% of tricalcium phosphate, 19% of microcrystalline cellulose, 2% of glyceryl palmitostearate, 0.6% of magnesium stearate and 4.7% of disintegrant was compressed together with a vitamin and mineral substance mixture in an E. Hata rotary tablet press give a bean-shaped tablet having a core weight of 1.0 g and the dimensions 18.0 mm×8.0 mm×7.2 mm. A mixture of 35 parts of hydroxypropylmethylcellulose and 60 parts of hydroxypropylcellulose and 5 parts of hydroxyethylcellulose was subsequently sprayed on from aqueous solution in a Bohle drum coater with a batch size of 5 kg. The amount of sprayed-on hydroxypropylmethylcellulose/hydroxypropylcellulose/hydroxyethylcellulose mixture was 5% by weight, based on the weight of the core, corresponding to 11.74 mg per cm² of tablet surface.

### EXAMPLE 8

A mixture of 9.8% of bacteria preparation, 35.0% of inulin, 28.7% of tricalcium phosphate, 18.9% of microcrystalline cellulose, 2.0% of glyceryl palmitostearate, 0.6% of magnesium stearate and 5.0% of disintegrant was compressed together with a vitamin and mineral substance mixture in an E. Hata rotary tablet press to give a bean-shaped tablet having a core weight of 1.0 g and the dimensions 18.0 mm×8.0 mm×7.2 mm. A mixture of 65 parts of hydroxypropylmethylcellulose and 35 parts of hydroxypropylcellulose was subsequently dissolved in water, and 5% by weight of magnesium stearate, based on the polymer dry substance, were subsequently incorporated, and the mixture was sprayed on in a Pellegrini drum coater with a batch size of 333 kg. The amount of sprayed-on hydroxypropylmethylcellulose/hydroxypropylcellulose mixture was 5% by weight, based on the weight of the core, corresponding to 11.74 mg per cm² of tablet surface.

1.-11. (canceled)

12. An oral administration form comprising a pharmaceutically acceptable carrier and at least one species of a probiotic microorganism, wherein the oral administration form and/or the probiotic microorganism is/are provided with a coating comprising at least two cellulose ethers which contain hydroxyalkyl groups as substituents
wherein said oral administration form does not contain a gastric juice resistant coating and does not contain a softener
and wherein the coating contains at least 35 parts of hydroxypropylcellulose.

13. An oral administration form according to claim 12, that is a tablet, a dragee, a capsule, a granular material or a powder.

14. An oral administration form according to claim 12, wherein the probiotic microorganism is Lactobacilli, Bifidobacteria or Streptococci.

15. An oral administration form according to claim 12, comprising 10³ to 10¹² probiotic microorganisms.

16. An oral administration form according to claim 12, wherein the hydroxyalkyl substituent group is hydroxyethyl, hydroxypropyl or dihydroxypropyl.

17. An oral administration form according to claim 16, wherein at least one of the cellulose ethers present in the coating also contains an alkyl group in addition to the hydroxyalkyl substituent group.

18. An oral administration form according to claim 17, wherein the coating comprises a cellulose ether which contains exclusively hydroxyalkyl groups as ether substituents and a cellulose ether which contains alkyl groups and hydroxyalkyl groups as ether substituents.

19. An oral administration form according to claim 18, wherein the cellulose ethers are hydroxypropylmethylcellulose and hydroxypropylcellulose.

20. An oral administration form according to claim 19, wherein the hydroxypropylmethylcellulose and the hydroxypropylcellulose are present in a weight ratio to one another of 90:10 to 10:90.

21. An oral administration form according to claim 12, further comprising a nutrition-relevant additive.

22. A process for the production of an oral administration form according to claim 12, comprising applying said coating from an aqueous solution and/or from an organic solution.

23. An oral administration form according to claim 15, comprising 10⁵ to 10¹¹ probiotic microorganisms.

24. An oral administration form according to claim 16, wherein hydroxy alkyl group is hydroxypropyl.

25. An oral administration form according to claim 13, wherein said form is a multilayered tablet.

26. An oral administration form according to claim 14, wherein the probiotic microorganism is Lactobacillus casei, Lactobacillus acidophilus, Lactobacillus reuteri, Lactobacillus bifidum, Lactobacillus gasseri, Lactobacillus plantarum, Lactobacillus johnsonii, Lactobacillus rhamnosus, Lactobacillus fermentum, Lactobacillus paracasei, Lactobacillus crispatus, Bifidobacterium longum, Bifidobacterium bifidum, Bifidobacterium longum, Bifidobacterium lactis, Bifidobacterium brevis, Bifidobacterium animalis, Bifidobacterium adolescentis, Bifidobacterium infantis, Streptococcus thermophilus or Lactococcus lactis.

27. An oral administration form according to claim 12, wherein the alkyl group is methyl or ethyl.

28. An oral administration form according to claim 12, wherein the hydroxypropylmethylcellulose and the hydroxypropylcellulose are present in a weight ratio of 70:30 to 30:70.

29. An oral administration form according to claim 28, wherein the hydroxypropylmethylcellulose and the hydroxypropylcellulose are present in a weight ratio of 35:65.

30. An oral administration form according to claim 21, wherein said nutrition-relevant additive is a vitamin, mineral, trace element, roughage, an enzyme, a plant extract, a protein, a carbohydrate or a fat.

31. A process according to claim 22, wherein said coating is applied from an aqueous solution.

32. An oral administration form according to claim 23, comprising 10⁷ to 10¹⁰ probiotic microorganisms.

33. An oral administration form comprising a pharmaceutically acceptable carrier and at least one species of a probiotic microorganism that is Lactobacilli, Bifidobacteria or Streptococci,
wherein the oral administration form and/or the probiotic microorganism is/are provided with a coating comprising hydroxypropylmethylcellulose, hydroxypropylcellulose and a stearate and said coating has a thickness of 5 to 15 mg per cm²
and wherein the coating contains at least 35 parts of hydroxypropylcellulose.

34. An oral administration form comprising a pharmaceutically acceptable carrier and at least one species of a probiotic microorganism, wherein the oral administration form and/or the probiotic microorganism is/are provided with a coating comprising
hydroxypropylmethylcellulose and hydroxypropylcellulose
and
wherein the survival rate of said probiotic microorganisms in the small intestine is at least 5-fold compared with the uncoated administration form
and wherein the coating contains at least 35 parts of hydroxypropylcellulose.

35. An oral administration form according to claim 12 wherein the proportion by weight of the cellulose ethers in the coating is 1.5 to 10% by weight, based on the total weight of the oral administration form.

36. An oral administration form according to claim 18 wherein said coating was spray applied from an aqueous solution.

37. An oral administration form comprising a pharmaceutically acceptable carrier and at least one species of a probiotic microorganism, wherein the oral administration form and/or the probiotic microorganism is/are provided with a coating comprising
hydroxypropylmethylcellulose and hydroxypropylcellulose in a weight ratio to one another of 90:10 to 10:90
wherein the coating contains at least 35 parts of hydroxypropylcellulose and
wherein said oral administration form does not contain a gastric juice resistant coating and does not contain a PEG softener.
